(19) United States
(12) Patent Application Publication
(10) Pub. No.: US 2018/0265868 A1
(43) Pub. Date: Sep. 20, 2018

(54) METHODS AND COMPOSITIONS COMPRISING APSE KNOTS

(71) Applicant: APSE, LLC, St. Louis, MO (US)

(72) Inventor: Juan P. H. ARHANCET, St. Louis, MO (US)

(73) Assignee: APSE, LLC, St. Louis, MO (US)

(21) Appl. No.: 15/763,431

(22) PCT Filed: Sep. 30, 2016
(86) PCT No.: PCT/US2016/054834

§ 371 Date: Mar. 26, 2018

Related U.S. Application Data
(60) Provisional application No. 62/235,992, filed on Oct. 1, 2015.

(51) Int. Cl. C12N 15/113

(52) U.S. Cl. CPC C12N 15/113; C12N 2330/50; C12N 2310/14

(57) ABSTRACT

The invention describes recombinant DNA sequences transcribed into RNA constructs capable of forming pseudoknots and being encapsidated in Virus Like Particles having higher insect control efficacy than previously described RNA molecules.

### CROSS REFERENCE TO RELATED APPLICATIONS

This application claims the benefit of U.S. Provisional Application No. 62/235,992 filed Oct. 1, 2015, the contents of which are incorporated herein by reference.

### INCORPORATION OF SEQUENCE LISTING

A Sequence Listing is provided herewith as a text file entitled “APSE_Knots_Sequence_Listing” created on Sep. 29, 2016 and having a size of 23 KB. The contents of the text file are incorporated by reference herein in their entirety.

### FIELD OF THE INVENTION

The invention described here involves methods and compositions for producing DNA constructs capable of generating RNA pseudoknots (APSE Knots) which can be processed by target host enzymes to produce effective RNAi-mediated gene suppression. Such compositions and methods have application in crop protection and insect control.

### BACKGROUND OF THE INVENTION

Recombinant RNA constructs used for RNAi purposes described in the prior art consist of double stranded RNAs (dsRNAs) between about 18 and about 25 base pairs (siRNAs), as well as longer dsRNA (long dsRNA) usually between 100 and about 1,000 base pairs (bp). Each of such constructs can consist of a pair of strands, sense and antisense, or of a single stranded hairpin where sense and antisense portions of the single strand are linked by a non-hybridizing sequence of at least about 100 nucleotides (nt), and most frequently at least a 150 nt sequence commonly referred to as a loop [Hauge et al. (2009) PLoS ONE 4(9): e7205]. The minimum dsRNA length needed for proper processing of such structures to active RNAi molecules by host enzymes ranges from approximately 33 bp in humans [Yates, et al. (2013) Cell , Volume 153, Issue 3, 516-519] to 37 bp and longer in plants [Song, et processes RNA hairpins comprising single stranded RNA (ssRNA) at one end of the dsRNA stem and a loop at the other end. However, bidirectional processing, i.e. processing from both ends of the dsRNA stem, has also been described in plants [Zhu, et al. (2013) Nature Structural & Molecular Biology 20, 1106-1115]. When dsRNA is used for insect control, dsRNAs longer than or equal to approximately 60 bp are sometimes required for efficient uptake when supplied in the insect's diet [Bolognesi, et al. (2012) PLoS One 7: e47534]. Long dsRNA molecules are cleaved in-vivo into a diverse population of siRNA molecules by the host's Dicer enzyme. While one or more of these siRNA molecules have the potential to be highly functional, thereby silencing the gene of interest, a significant fraction are non-functional, i.e. induce little or no silencing [Khvorova, et al. U.S. Pat. No. 8,090,542]. There remains a need for long dsRNA molecules that are cleaved into a large fraction of highly functional siRNA molecules.

Double stranded RNA is sensitive to degradation by nucleases in the host, which reduce its RNAi efficacy. Sections of transcribed strands used to reduce degradation of long dsRNA by nucleases have been described. For example, pseudoknots have been used for this purpose [Allen, et al. U.S. Patent Application 20070011775]. Pseudoknots are RNA constructs comprised of sense/antisense sections having at least one base pair, ii* with bases in positions i and i* within the RNA strand, where i* is complementary to i, and another at least one base pair, jj* in the same RNA strand as ii*, in positions j and j*, where j* is complementary to j, and where position i<j<i*<j* within the molecule, as defined by Achawanantakun and Sun (2013) BMC Bioinformatics, 14 (Suppl 2):S1. Different types of pseudoknots have been described in prior art, e.g. by Staple and Butcher, PLoS Biology June 2005, 3 (6):0956. Sense/antisense stems in pseudoknots described in the prior art are shorter than 15 bp, and do not elicit an active RNAi response. We describe here a form of RNA pseudoknot that comprises sense/antisense stems sufficient to and arranged in such a manner as to elicit an active RNAi response upon processing by host Dicer enzymes, such RNAs are referred to here as APSE Knots.

In addition to the problems associated with degradation by host nucleases, RNA is also subject to a variety of non-specific nucleases and degradative processes both within the host and in the environment generally. APSE has pioneered methods of packaging RNA within bacteriophage capsids to form Virus Like Particles (VLPs) comprising an active heterologous RNA protected from most environmental and host nuclease activities as long as the capsid of the VLP remains intact. The ability of VLPs to facilitate production and stability of large quantities of ssRNA and dsRNA are discussed in WO 2015/038915 and WO 2013/096866, the contents of each which are incorporated in their entirety herein by reference.

### SUMMARY OF THE INVENTION

The invention described here uses the unique properties of APSE Knots, optionally as VLPs, to provide an improved system for delivering active RNAi substrates to suppress expression of a target gene, preferably in an insect host, more preferably a Coleopteran or Lepidopteran insect pest. Of particular interest are Coleoptera such as bark beetle, elm leaf beetle, Asian longhorn beetle, death watch beetle, mountain pine beetle, coconut hispine beetle and the Colorado potato beetle. RNAi methods of controlling Colorado potato beetle are especially desired since these beetles have developed resistance to virtually all known insecticides. Leptidoptera of particular interest include, without limitation, army worms, corn ear worm, corn rootworm, cabbage butterfly and cotton boll worm. Of special interest is effective RNAi control of corn rootworms, which have historically been impervious to such treatment due to a relative (to other insect) insensitivity to RNAi methods.

Both Coleopteran and Lepidopteran insect pests are known to be susceptible to RNAi introduced via the gut, either by direct injection or by feeding on plant matter treated with siRNA precursors. Field application of naked RNAs is generally impractical due to the sensitivity of RNA to environmental specific and non-specific degradation. Furthermore, RNA is highly susceptible to degradation during the course of feeding and in transit through the insect gut. In general the Lepidoptera seem to degrade RNA much more aggressively than the Coleoptera, which may account for their relatively poor susceptibility to RNAi mediated control methods. The highly compact structural form of APSE Knots reduces succeptibility to such specific and non-specific RNA degradation. In one embodiment the present invention further circumvents this limitation by packaging the RNAi precursor molecule within a VLP, which serves to protect the RNA from host nucleases and limits non-specific environmental degradation.

Another major advantage of producing RNAi by the methods described here is that costly and complicated in vitro synthesis of RNA precursors is avoided and the desired RNA constructs can be produced by simple and economic fermentation methods. Production and purification of large quantities of RNAi precursors is facilitated by optionally coupling synthesis of the desired polynucleotide with expression of self-assembling bacteriophage capsid proteins, such as those of bacteriophage Qβ or MS2 to produce easily purified and relatively stable VLPs, which may be applied directly to plant surfaces upon which the targeted insect pests feed, for example by spraying.

Once ingested, the VLPs may be digested in the course of transiting the insect host gut and the RNA molecules absorbed by cells lining the gut, where they can be processed by, among other things, the host Dicer enzyme to generate effective RNAi targeted against host gene transcripts to suppress expression of essential host genes. Examples of such essential genes include genes involved in controlling molting or other larval development events, actin or other cellular structural components, as well as virtually any gene essential to viability of the target insect pest.

The inventors have discovered that RNA constructs of the type 5′-. . . -A-B-C- . . . -A*-B*-C*- . . . -3′ where A, B, C are sense sequence and A*, B*, C* are their respective antisense sequences, for example A* being antisense to A and B* being antisense to B, are produced efficiently by fermentation and have higher RNAi efficacy than dsRNA described in prior art. RNA constructs of the instant invention constitute a subclass of pseudoknots. The inventors have found that efficient production by fermentation can be achieved when sense/antisense sequence pairs are separated by at least 40 nt and such constructs are effective in RNAi applications. Furthermore, effective RNAi response can be achieved when immediately adjacent strands are separated by less than 10 nucleotides.

The RNA molecules described here as APSE knots possess a number of useful and unusual features. Junk RNA, RNA not useful for RNAi activity, is reduced by reducing the size of the loop in the transcribed RNA hairpin structure by replacing the conventional loop with sense or anti-sense RNA strands containing linkers of 10 nucleotides or less. The amount of junk RNA is also reduced by minimizing the sequences flanking the transcribed RNAi sequences using small self-cleaving ribozyme sequences. Such RNA molecules can be used directly in insect control applications or they can be incorporated into VLPs prior to use in controlling insect pests. Within the overall process of packaging and VLP purification, contaminating bacterial host RNA is minimized by the inherent packaging preference to molecules containing specific bacteriophage pac sequences and the conditions used to purify the VLPs containing the specifically packaged RNAs. The ability of sequences bearing pac sites to preferentially occupy VLPs is increased by lowering the number of pac sites per target transcript. In addition, placing the pac site at the 3′ end of the desired target transcript minimizes packaging of incomplete transcripts, maximizes the ability of transcripts to self-hybridize and thereby assume a more compact configuration which accelerates RNA packaging and allows longer transcripts to be packaged. Further, APSE Knots are designed to place the 5′ and 3′ ends of the RNA within the interior of the structure making them less accessible to exonucleases and positioning them along one stem of the APSE Knot. The configuration of the APSE Knot reduces the length of dsRNA accessible to Dicer so that the necessary cuts required to produce the desired RNAi precursor are guided to the optimal positions in the APSE Knot.

### DESCRIPTION OF THE DRAWINGS

FIG. 1 represents an APSE Knot comprising a sense RNA strand (section s) with 3 contiguous sections 72 nucleotides each, and 4 antisense sections, 13 nucleotides (section a), 2 sections of 42 nucleotides each (sections b & c) and 59 nucleotides (section a′). Numbers delineating each section correspond to nucleotide positions in sequence described in example DNA-AK72x3 (SEQ ID NO.: 1). The left hand panel represents the linear relationship of the sections to one another; the right hand panel represents how the sense and anti-sense pairings take place in two- and three-dimensional space.

FIG. 2 represents an APSE Knot comprising a sense RNA strand (section s) with 8 contiguous sections 36 nucleotides each, and 9 antisense sections 13 nucleotides (section a), 7 sections of 36 nucleotides each (sections b through h), and 23 nucleotides (section a′). Numbers qualifying each section correspond to nucleotide positions in sequence described in example DNA-AK36x8 (SEQ ID NO.: 4. The left hand panel represents the linear relationship of the sections to one another; the right hand panel represents how the sense and anti-sense pairings take place in two- and three-dimensional space.

### DETAILED DESCRIPTION OF THE INVENTION

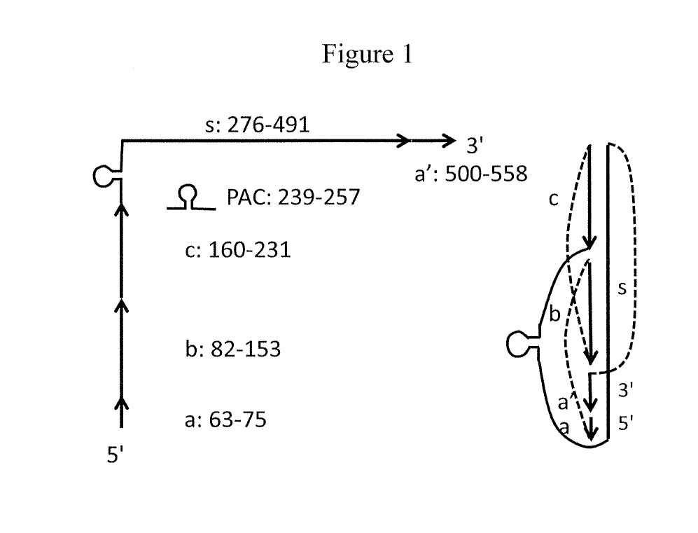

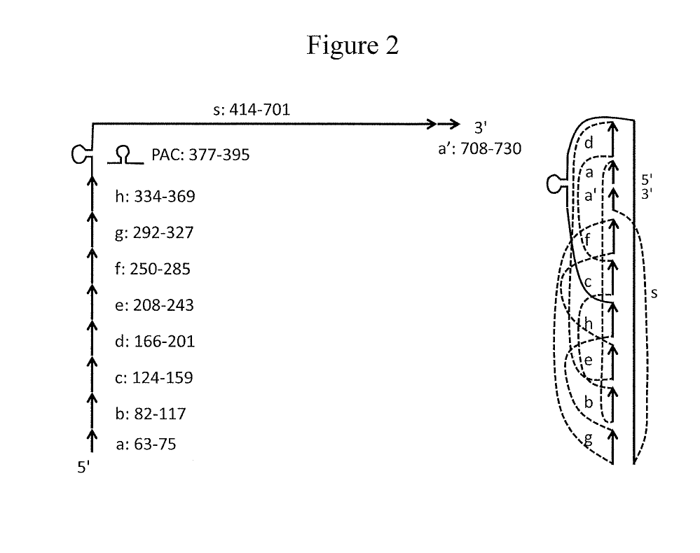

The present invention comprises DNA sequences, which when transcribed produce RNAi precursor molecules with uniquely stable structures suitable for packaging within bacteriophage capsids to form VLPs. A key feature of these RNA molecules is that they form a pseudoknot comprising multiple contiguous sense/antisense stems as depicted in FIGS. 1 and 2. Such structures are referred to here as APSE Knots. VLPs containing such APSE Knots can be ingested by insect larvae thereby introducing the APSE Knots into the insect gut where they can be taken up by host insect cells and processed into RNAi-effective forms capable of inhibiting growth or killing the insect host. Example sequences presented here are designed to be ligated into suitable bacterial plasmid vectors as AsiSI-NotI fragments. Such DNA sequences can be produced by direct synthesis or by sub-cloning the constituent fragments well known to those skilled in the art. The specific sequences may be modified as desired to manipulate specific restriction enzyme sites, incorporate alternative ribozymes, accommodate alternative bacteriophage pac sequences, or to modify the specificity of the RNAi sequences to target different genes and insect hosts. Bacterial plasmid vectors containing transcriptional promoters capable of inducibly transcribing these DNA sequences include without limitation, bacteriophage T7 gene 1 promoter, bacteriophage T5 promoter and the bacteriophage lambda P_{L} and P_{R} promoters. Bacterial plasmid vectors may also contain the bacteriophage Qβ or bacteriophage MS2 capsid protein coding sequence expressed from an inducible promoter. Alternatively, such inducibly expressed capsid proteins may be present on a separate bacterial plasmid compatible with the bacterial plasmid carrying the inducible cargo RNA sequences.

The production and purification of VLPs containing such cargo molecules are described in detail in WO 2015/038915 and WO 2013/096866. The VLPs produced by these methods can be processed in a number of different ways known to those skilled in the art to facilitate application of such material onto plants and for use in the field.

A person skilled in the art understands that the Examples presented here may be modified to target different genes in different insect hosts by modifying the sequences from those described to reflect the sequences of the targeted genes in the targeted host organisms. In addition to targeting specific sequences within a target gene for RNAi-mediated gene suppression, the ability to direct Dicer to cut at specific points or within a limited range of positions within an APSE Knot, by manipulating the size, extent and location of dsRNA regions within the APSE Knot, allows rapid identification of the specific sequences defining the most effective RNAi target within the target gene of a selected organism. Thus, APSE Knots provide those skilled in the art with a tool for identifying the best RNAi target for suppressing a particular gene in any given host cell and a means for producing large quantities of such RNAis.

### Preferred Embodiments

In one embodiment of the present invention, a DNA sequence within a bacterial host is transcribed to produce an RNA molecule comprising a Hammerhead ribozyme followed by a series of short contiguous antisense sequences based on those of a host insect target gene, followed by a bacteriophage pac site, followed by the sense sequence of the host insect target sequence, a single additional short antisense sequence to the host insect target sequence, which is turn followed by an HDV ribozyme. This RNA molecule, referred to here as an APSE Knot, is optionally processed and packaged within a VLP produced in the bacterial host and is isolated and purified prior to application to the outer surfaces of a plant. Target insects feeding upon that plant ingest the APSE Knot which in turn is introduced into host insect cells where it is processed by the host cell's Dicer pathway, resulting in RNAi-mediated suppression of gene expression of the host insect target gene.

In another embodiment, a series of DNA sequences as described in the previous paragraph are transcribed and may be packaged in VLPs. The DNA sequences within the series each encode a different set of short contiguous antisense sequences based on the host insect target gene. The series is designed so that the length and distribution of the short antisense sequences produces a different APSE Knot for each of the transcribed DNA sequences within the series. Each of the different APSE Knots is processed by the host insect Dicer pathway to produce a limited set of RNAi precursors from each APSE Knot. The APSE Knots are purified and fed to host insects; those producing the greatest level of RNAi-mediated suppression of gene expression represent the best RNAi target for that particular host insect target gene. Recourse to the corresponding bacterial cell line carrying the identified DNA sequence encoding the most effective APSE Knot allows quick scale-up of the desired APSE Knot for RNAi-mediated suppression of gene expression of the host insect target gene or further experimental investigation.

### EXAMPLE 1

### Efficacy of Insect Control by VLPs Containing an RNAi Precursor.

The ability of the methods described here to effectively deliver RNAi precursors to host insects is tested by constructing a DNA sequence possessing all of the preferred embodiments described here but with a known dsRNA RNAi precursor sequence in place of the APSE Knot, and processing and feeding the resulting VLPs to western corn rootworm. DNA construct DNA-HP235-150 (SEQ ID NO.: 9) which contains the sequence reported by Bolognesi et al. to produce a 21-mer RNAi precursor effective in suppressing expression of the western corn rootworm (Diabrotica virgifera virgifera) Snf7 ortholog, DvSnf7, when fed to the host insects. The western corn rootworm DvSnf7 gene encodes a critical component of the organism's endosomal sorting complex (ESCRT-III) and significant suppression of this essential gene results in larval death. DNA-HP235-150 (SEQ ID NO.: 9), and all other constructs described here, are cloned into a pBR322-based plasmid containing a T7 promoter, a multi-cloning site possessing AsiSI and NotI restriction sites, and a copy of the bacteriophage MS2 capsid protein, oriented such that T7 polymerase transcribes both any cloned AsiSI-NotI fragment inserted into the plasmid and the MS2 capsid protein gene. The plasmid is transformed into E. coli host strain HTE115(DE3) and ampicilin selected clones grown at 37° C. in LB media containing ampicilin until the culture reaches OD600 0.8, at which time isopropyl β-D-thiogalactopyranoside is added to a final concentration of 1 mM to induce expression of T7 polymerase. The induced cultures are harvested 4 hours post-induction by centrifugation at 3,000 g at 4 C. Each pellet is stored at 4° C. until processing.

Briefly, VLPs containing DNA-HP235-150 (SEQ ID NO.: 9), as well as the DNA constructs described in subsequent Examples here, are purified by re-suspending each pellet in approximately 10 volumes of 20 mM Tris-HCl, pH 7.0, containing 10 mM NaCl and sonicated to lyse the cells. Cell debris is removed by centrifugation at 16,000 g. Each sample is further processed by addition of Benzonase® Nuclease (Sigma Aldrich, St. Louis, Mo.) added to a final concentration of 100 units per mL and incubated at 37° C. for two hours. Proteinase K is then added to final concentration of 150 micrograms per mL and incubated at 37° C. for an additional three hours. At this point the VLP samples are ready for fractional ammonium sulfate precipitation. Fractional precipitation of VLPs is conducted as follows. A saturated ammonium sulfate solution is prepared by adding ammonium sulfate to water to a final concentration of 4.1 M. The saturated ammonium sulfate is added to the enzymatically treated VLPs to a final concentration of 186 mM (approximately a 1:22 dilution) and placed on ice for two hours. Unwanted precipitate is cleared from the lysate by centrifugation at 16,000 g. Each sample is then subjected to a second precipitation by the addition of 155 mg of dry ammonium sulfate directly to each mL of cleared lysate. Each sample is vortexed and incubated on ice for two hours. Each precipitate is spun down at 16,000 g and the solid precipitate is kept and resuspended in one tenth the original volume of 20 mM Tris-HCl, pH 7.0, containing 10 mM NaCl.

Bioassays are performed using a diet overlay methodology. Commercial western corn rootworm diet is prepared according to manufacturer's guidelines for SCR diet (Bio-Serv, Frenchtown, N.J.) with a few adjustments as described by Bolognesi et al., including the addition of Formalin at 0.06% (v/v), 10% KOH (v/v) to increase pH to 9, and lyophilized corn root tissue at 0.62% (w/v). Two hundred μl of molten diet is pipetted into 24 wells of 96 well plates (Falcon), and allowed to solidify at room temperature. Known amounts, ranging from 0.5 ng to 500 mgs of VLPs containing the DNA-HP235-150 (SEQ ID NO.: 9) RNA and control samples comprising approximately 2-200 ng of unencapsidated RNA from DNA-HP235-150 (SEQ ID NO.: 9) is overlaid in each well. Controls include the 240 base pair RNA molecule described by Bolognesi et al. as effective in killing western corn rootworm by suppression of the DvSnf7 gene (positive control) and a VLP comprising RNA sequences entirely unrelated to western corn rootworm (negative control). Plates are air dried and one larva is added per well. Plates are sealed with Mylar, ventilation holes added to each well with a #1 or #2 insect pin, and the plates incubated at 27° C. for 12 days. A cohort of 10 larva are fed each individual DvSnf7 APSE Knot construct or control sequence to provide ten data points for each experimental sample or control. Growth inhibition (larval size assessed from daily pictures) and mortality was determined for each cohort.

Each experimental and control cohort within the experiment is comprised of 10 individual larva undergoing 10 identical treatments. Since the only way to ensure that an individual larva has consumed an entire dose, each larva is dosed in isolation. Any larvae that die in the course of the experimental procedure are processed to recover total mRNA and the sample preserved at −80° C. until further analysis can take place.

Once the 12 day experimental period is completed, the growth rate and overall mortality of each cohort is assessed and the remaining live larvae sacrificed and total mRNA recovered. The naked dsRNA treated controls exhibit a high degree of mortality, consistent with the observations of Bolognesi et al., that suppression of DvSnf7 gene expression by this dsRNA results in death of larvae that consume it. The cohort treated with VLPs containing the unrelated RNA exhibit little or no mortality, indicating that VLPs are not inherently toxic to the larvae. Increasing mortality of larvae in the cohorts consuming the VLPs containing the DNA-HP235-150 (SEQ ID NO.: 9) version of the dsRNA of Bolognesi et al. indicates that the VLPs provide an effective delivery platform for such molecules and verifies that the packaging and processing steps for manufacturing VLPs does not inhibit effectiveness of the RNAi response observed from such dsRNA.

In all cases, the mRNA samples are analyzed by quantifying expression of the actin gene relative to standard markers and the results compared with the mortality rates exhibited by each experimental cohort. Reduced intact DvSnf7 mRNA indicates effective RNAi suppression of gene expression. Intact DvSnf7 mRNA can be measured by qPCR, qrtPCR, by differential Northern blot analysis or by similar quantitative methods.

### EXAMPLE 2

### Effect of Stem Length and Number on RNAi Activity of APSE Knots

To determine whether RNAi precursors lacking the 150 nucleotide loop present in the structure reported by Bolognesi et al., might exhibit greater stability and to increase the ability to pack more such molecules within a single VLP, a series of constructs is designed to omit the loop sequence entirely and break the antisense stem into 4 smaller stems with short 7 nucleotide intervening sequences between three of the four antisense segments, with a fourth segment distal to the sense sequence. In the process, the basic APSE Knot format is produced as diagrammatically outlined in FIG. 1.

DNA constructs, DNA-AK72x3 (SEQ ID NO.: 1), DNA-AK42x5 (SEQ ID NO.: 2), and DNA-AK30x7 (SEQ ID NO.: 3), describe DNA sequences coding for RNAs representing APSE Knots having an odd number of stems of different lengths, 72 bp, 42 bp and 30 bp respectively, with approximately the same total length of RNA antisense to a target actin gene of the Colorado potato beetle (Leptinotarsa decemlineata strain Freeville actin mRNA, GenBank sequence ID: gb|KJ577616.1) of 216 nt, 210 nt, and 210 nt respectively. In each case, one of the stems is formed by one uninterrupted strand and two reverse complementary strands, one proximal and one distal to the 5′ end of the molecule (as shown in FIG. 1). Each of the DNA constructs was cloned, induced and VLPs recovered as described in Example 1. The relative effectiveness of these three examples in killing Colorado potato beetles by suppressing expression of the essential actin gene, as described in Example 1, shows that APSE Knots with different configurations can target the same gene, depending on the particular length of contiguous nucleotides that may need to be targeted. In each of these constructs the sense strands (i, j, k, . . . ) are arranged in the same order as corresponding antisense strands (i*, j*, k*, . . . ), i.e. 5′-i, j, k, . . . , z, i*, j* k*, . . . , z*-3′ where there is an odd number of sense strands and z represents the largest number of total sense/antisense sequences within the APSE Knot.

DNA construct, DNA-AK36x8 (SEQ ID NO.: 4), encodes an RNA APSE Knot directed against the actin gene of the Colorado potato beetle with an even number of stems, 8 in this case, in which the antisense strands are arranged in a different order than the sense strands (as shown in FIG. 2). This is necessary to keep at least 150 nucleotides between all corresponding pairs of sense/antisense strands.

The VLPs containing each of the DNA constructs described in this Example 2 are applied in 50 microliter droplets to the surface of a 2 cm diameter leaf disc punched from a potato leaf. A 1 cm disc can be used for early larval stage if necessary. The solution is spread with the pipette tip to cover at least the central half of the leaf disc. The insect will devour all of the leaf tissue, without veins. Leaf discs are placed in a petri dish and the treatment liquid allowed to dry on the leaf surface. After the treatment liquid has dried one Colorado potato beetle larvae is applied to the leaf disc. After the larvae have devoured the entire leaf disc the remaining vein tissue is removed from the petri dish and the beetle is fed more potato leaves or an artificial diet. The beetle larvae are starved for 2-24 hours before dosing. The starvation period is partially determined by whether the maintenance diet is either potato leaves or artificial diet. Post dosing beetle larvae remain in the same petri dish (veins from dosing disc are removed). Three hours post dosing beetle larvae are returned to a maintenance diet of either potato leaves or artificial diet. Beetles are dosed three times for each treatment, dose 1 is delivered on day 1, dose 2 is delivered on day 3, and dose 3 is delivered on day 5. Post-dosing, beetle larvae are not fed for 2-24 hours and are then placed on a maintenance diet of potato leaves or artificial diet until prior to the next dosing cycle. Following the final dose and post dose starvation period beetles are maintained on either potato leaves or artificial diet for 21 days. Mortality of the beetles is recorded for each sample.

The experimental samples comprise increasing concentrations of VLPs, each containing APSE Knots from DNA-AK72x3 (SEQ ID NO.: 1), DNA-AK42x5 (SEQ ID NO.: 2), DNA-AK30x7 (SEQ ID NO.: 3 and DNA-AK36x8 (SEQ ID NO.: 4), as well as a negative control comprising high concentration of a VLP containing RNA sequences unrelated to Colorado potato beetle. Each experimental and control cohort includes 10 individual beetles undergoing 10 identical treatments. Since the only way to ensure that an individual beetle has consumed an entire dose, each beetle is dosed in isolation. Any beetles that die in the course of the experimental procedure are processed to recover total mRNA and the sample preserved at −80° C. until further analysis can take place.

Once the 26 day experimental period is completed, the overall mortality of each cohort is assessed and the remaining live beetles sacrificed and total mRNA recovered. The naked dsRNA treated controls exhibit a high degree of mortality, consistent with the observations of Bolognesi et al., that suppression of actin gene expression by this dsRNA results in death of beetles that consume it. The cohort treated with VLPs containing the unrelated RNA exhibit little or no mortality, indicating that VLPs are not inherently toxic to the beetles. Increasing mortality of beetles in the cohorts consuming the VLPs containing the DNA-AK72x3 (SEQ ID NO.:1), DNA-AK42x5 (SEQ ID NO.: 2), DNA-AK30x7 (SEQ ID NO.: 3 and DNA-AK36x8 (SEQ ID NO.: 4) APSE Knots indicates that the VLPs provide an effective delivery platform for such molecules and verifies that the packaging and processing steps for manufacturing VLPs does not inhibit effectiveness of the RNAi response observed from such dsRNA.

In all cases, the mRNA samples are analyzed by quantifying expression of the actin gene relative to standard markers and the results compared with the mortality rates exhibited by each experimental cohort. Reduced intact mRNA specific for actin indicates effective RNAi suppression of gene expression. Intact actin mRNA can be measured by qPCR, qrtPCR, by differential Northern blot analysis or by similar quantitative methods.

The ability of each of these constructs to kill Colorado potato beetle confirms that the basic APSE Knot configuration is an effective tool for producing targeted RNAi precursors into an insect host and that these precursors can be properly processed by the host cell Dicer pathway to suppress gene expression of the target gene.

### EXAMPLE 3

Effect of Stems with Differing Length on RNAi Activity of APSE Knots

To test the flexibility of incorporating stems of differing length within a single APSE Knot, DNA constructs DNA-AK43x5 (SEQ ID NO.: 5), DNA-AK45x5 (SEQ ID NO.: 6), DNA-AK47x5 (SEQ ID NO.: 7) and DNA-AK49x5 (SEQ ID NO.: 8) are produced and cloned and packaged into corresponding VLPs as described above. Each of the constructs contains 5 stems, as indicated by the last number in the construct name of 43, 45, 47 or 49 nucleotides, as indicated by the first number within the construct name. Comparison of the ability of these constructs to kill western corn rootworm, measured as described in Example 1, indicates that APSE Knots with different stem lengths containing the same 21 nucleotide dsRNA can be used to target the same gene, depending on the minimum stem length of dsRNA needed by host Dicer processing the APSE Knot.

This observation allows practitioners to devise a series of APE Knots with different stem lengths having different joints between the antisense portion of each stem to probe the position of the most effective RNAi precursor within a given target gene, merely by changing stem length within a series of APSE Knots based on the target gene sequence. Host cell Dicer enzymes are sensitive to the stem length of dsRNA substrates and by manipulating the length and position of the sense/antisense structures within the pseudoknot structure Dicer can be forced to make only one or a limited number of cuts within any given APSE Knot. A series of APSE Knots with differing sense' antisense structures can be assessed for RNAi activity allowing rapid identification of the most effective RNAi target sequence within the gene upon which the APSE Knot series is based.

1. A recombinant DNA construct capable of producing an RNA comprising a sense strand i, a sense strand j, a strand i* antisense to i, and a strand j* antisense to j, each strand being between 27 nt and 1000 nt long, where such strands are located in order 5′-i-j-i*-j*-3′.

2. The RNA of claim 1 wherein the 3′ end of at least one of the sense strands is separated by at least 40 nucleotides from the 5′ end of its corresponding antisense strand.

3. A composition comprising the RNA of claim 2 and at least one viral capsid protein.

4. The composition of claim 3 wherein the viral capsid protein comprises the coat protein of bacteriophage MS2.

5. The composition of claim 3 wherein the viral capsid protein comprises the coat protein of bacteriophage Qβ.

6. The RNA of claim 1 further comprising a sense strand k and a strand k* antisense to k, with strands k and k* being between 27 nt and 1000 nt long, where such strands are located in the order 5′-i-j-k-i*-j*-k*-3′.

7. The RNA of claim 6 wherein the 3′ end of at least one of the sense strands is separated by at least 40 nucleotides from the 5′ end of its corresponding antisense strand.

8. The RNA of claim 7 wherein at least two immediately adjacent strands chosen from said positions i through k* are separated by not more than 10 nucleotides.

9. The RNA of claim 8 where at least one of the strands has at least one 18 nucleotide section with at least 95% homology to a section of RNA produced in a target host cell to which such construct is intended to be delivered.

10. A composition comprising the RNA of claim 6 and at least one viral capsid protein.

11. The composition of claim 10 wherein the viral capsid protein comprises the coat protein of bacteriophage MS2.

12. The composition of claim 10 wherein the viral capsid protein comprises the coat protein of bacteriophage Qβ.


SEQUENCE LISTING (9 sequences)

SEQ ID NO: 1 - DNA, 632 bp, Artificial Sequence
  DNA sequence designed for transcription into an RNA.
  misc_feature (1)..(8): AsiSI restriction site
  misc_feature (9)..(62): Hammerhead ribozyme, cuts between residues 62 and 63.
  misc_feature (63)..(75): antisense to sequence between residues 276-288.
  misc_feature (82)..(153): antisense to sequence between residues 348-419.
  misc_feature (160)..(231): antisense to sequence between residues 420-491.
  misc_feature (239)..(257): bacteriophage MS2 PAC site.
  misc_feature (268)..(275): PmeI restriction site.
  misc_feature (276)..(491): nucleotides 46-261 of  Leptinotarsa decemlineata strain Freeville actin mRNA, Genbank sequence ID gb|KJ577616.1.
  misc_feature (492)..(499): PacI restriction site.
  misc_feature (500)..(558): antisense to sequence between residues 289-347.
  misc_feature (559)..(624): HDV ribozyme, cuts between residues 558 and 559.
  misc_feature (625)..(632): NotI restriction site.
gcgatcgcatcaacatgtgtgacctgatgaggccgaaaggccgaaacccgtgaaaacgggtcgtcacacatgttgatccctccgaccatgactccttgatgccttgggcgaccgacgatcgaggggaagacggcacggggtgcgtcatctcctatccctgggtatttcagggtgaggatacctcttttgctttgggcttcatctcctacgtatgagtccttttgtcccataatccctaacatgaggattacccatgtaactaactaagtttaaaccaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaatacccttaattaagcgaaaccggctttgcacataccggatccattgtctacgacaagagccgctacatcgtcggccggcatggtcccagcctcctcgctggcgccggctgggcaacattcgtggcgaatgggaccatagcggccgc

SEQ ID NO: 2 - DNA, 632 bp, Artificial Sequence
  DNA sequence designed for transcription into an RNA
  misc_feature (1)..(8): AsiSI restriction site.
  misc_feature (9)..(62): Hammerhead ribozyme cuts between residues 62-63.
  misc_feature (63)..(75): antisense to sequence between residues 312-324.
  misc_feature (82)..(123): antisense to sequence between residues 354-395.
  misc_feature (130)..(171): antisense to sequence between residues 396-437.
  misc_feature (178)..(219): antisense to sequence between residues 438-479.
  misc_feature (226)..(267): antisense to sequence between residues 480-521.
  misc_feature (275)..(293): bacteriophage MS2 PAC site.
  misc_feature (304)..(311): PmeI restriciton site.
  misc_feature (312)..(521): nucleotides 16-225 of  Leptinotarsa decemlineata strain Freeville actin mRNA, Genbank sequence ID gb|KJ577616.1.
  misc_feature (522)..(529): PacI restriction site.
  misc_feature (530)..(5558): antisense to sequence between residues 325-353.
  misc_feature (559)..(624): HDV ribozyme cuts between residues 558-559.
  misc_feature (625)..(632): NotI restriction site.
gcgatcgcatgtctagtgagcagctgatgaggccgaaaggccgaaacccgtgaaaacgggtcctgctcactagacatccctataccggatccattgtctacgacaagagccgctacatcgtcgatccctacggcacggggtgcgtcatctcctgcgaaaccggctttgcacatccctatgactccttgatgccttgggcgaccgacgatcgaggggaagatccctgcttcatctcctacgtatgagtccttttgtcccataccgaccatccctaacatgaggattacccatgtaactaactaagtttaaacgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcttaattaatcacacatgttgtcttttgaggttggacaggccggcatggtcccagcctcctcgctggcgccggctgggcaacattcgtggcgaatgggaccatagcggccgc

SEQ ID NO: 3 - DNA, 642 bp, Artificial Sequence
  DNA sequence designed for transcription into an RNA.
  misc_feature (1)..(8): AsiSI restriction site.
  misc_feature (9)..(62): Hammerhead ribozyme cuts between residues 62-63.
  misc_feature (63)..(75): Hammerhead ribozyme cuts between residues 62-63.
  misc_feature (63)..(75): antisense to sequence between residues 336-348.
  misc_feature (82)..(111): antisense to sequence between residues 366-395.
  misc_feature (118)..(147): antisense to sequence between residues 396-425.
  misc_feature (154)..(183): antisense to sequence between residues 426-455.
  misc_feature (190)..(219): antisense to sequence between residues 456-485.
  misc_feature (226)..(255): antisense to sequence between residues 486-515.
  misc_feature (262)..(291): antisense to sequence between residues 516-545.
  misc_feature (299)..(317): bacteriophage MS2 PAC site.
  misc_feature (328)..(335): PmeI restriction site.
  misc_feature (336)..(545): nucleotides 4-213 of  Leptinotarsa decemlineata strain Freeville actin mRNA, Genbank sequence ID gb|KJ577616.1.
  misc_feature (552)..(568): antisense to sequence between residues 349-365.
  misc_feature (569)..(634): HDV ribozyme cuts between residues 558-559.
  misc_feature (635)..(642): NotI restriction site.
gcgatcgcatcgaggtttttctgctgatgaggccgaaaggccgaaacccgtgaaaacgggtccagaaaaacctcgatcccttcgtcgtcacacatgttgtcttttgaggttatccctgatccattgtctacgacaagagccgctacaatcccttctcctgcgaaaccggctttgcacataccgatccctatcgaggggaagacggcacggggtgcgtcaatccctatgactccttgatgccttgggcgaccgacgatccctacgtatgagtccttttgtcccataccgaccatccctaacatgaggattacccatgtaactaactaagtttaaaccgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtatccctggacactgctcactagaggccggcatggtcccagcctcctcgctggcgccggctgggcaacattcgtggcgaatgggaccatagcggccgc

SEQ ID NO: 4 - DNA, 804 bp, Artificial Sequence
  DNA sequence designed for transcription into an RNA.
  misc_feature (1)..(8): AsiSI restriction site.
  misc_feature (9)..(62): Hammerhead ribozyme cuts between residues 62-63.
  misc_feature (63)..(75): antisense to sequence between residues 450-462.
  misc_feature (82)..(117): antisense to sequence between residues 630-665.
  misc_feature (124)..(159): antisense to sequence between residues 522-557.
  misc_feature (166)..(201): antisense to sequence between residues 414-449.
  misc_feature (208)..(243): antisense to sequence between residues 594-629.
  misc_feature (250)..(285): antisense to sequence between residues 486-521.
  misc_feature (292)..(327): antisense to sequence between residues 666-701.
  misc_feature (334)..(369): antisense to sequence between residues 558-593.
  misc_feature (377)..(395): bacteriophage MS2 PAC site.
  misc_feature (406)..(413): PmeI restriction site.
  misc_feature (414)..(701): nucleotides 10-297 of  Leptinotarsa decemlineata strain Freeville actin mRNA, Genbank sequence ID gb|KJ577616.1.
  misc_feature (708)..(730)
  misc_feature (708)..(730): antisense to sequence between residues 463-485.
  misc_feature (731)..(796): HDV ribozyme cuts between residues 730-731.
  misc_feature (797)..(804): NotI restriction site.
gcgatcgcatcaacatgtgtgacctgatgaggccgaaaggccgaaacccgtgaaaacgggtcgtcacacatgttgatccctgggtatttcagggtgaggatacctcttttgctttggatccctatcgaggggaagacggcacggggtgcgtcatctcctatcccttcttttgaggttggacactgctcactagacagaaaaatccctgcttcatctcctacgtatgagtccttttgtcccataatccctgcgaaaccggctttgcacataccggatccattgtctatccctatgtcatcccagttggtgatgataccgtgttcgatgatccctccgaccatgactccttgatgccttgggcgaccgacgatccctaacatgaggattacccatgtaactaactaagtttaaacttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgacatatccctacgacaagagccgctacatcgtcggccggcatggtcccagcctcctcgctggcgccggctgggcaacattcgtggcgaatgggaccatagcggccgc

SEQ ID NO: 5 - DNA, 642 bp, Artificial Sequence
  DNA sequence designed for transcription into an RNA
  misc_feature (1)..(8): AsiSI restriciton site.
  misc_feature (9)..(62): Hammerhead ribozyme cuts between residues 62-63.
  misc_feature (63)..(75): antisense to sequence between residues 316-328.
  misc_feature (82)..(124): antisense to sequence between residues 359-401.
  misc_feature (131)..(173): antisense to sequence between residues 402-444.
  misc_feature (180)..(222): antisense to sequence between residues 445-487.
  misc_feature (229)..(271): antisense to sequence between residues 488-530.
  misc_feature (279)..(297): bacteriophage MS2 PAC site.
  misc_feature (308)..(315): PmeI restriciton site.
  misc_feature (316)..(412): filler sequence.
  misc_feature (413)..(433): sequence no. 21.3 of Bolognesi, et al., (2012) PLoS One 7e47534.
  misc_feature (434)..(530): filler sequence.
  misc_feature (531)..(538): PacI restriction site.
  misc_feature (539)..(568): antisense to sequence between residues 329-358.
  misc_feature (569)..(634): HDV ribozyme, cuts between residues 568 and 569.
  misc_feature (635)..(642): NotI restriction site.
gcgatcgcatactaaccaaaagcctgatgaggccgaaaggccgaaacccgtgaaaacgggtcgcttttggttagtatccctctacaaaagtatctcactcaggcactgagctttctctgtcgtgatccctcttagatgtacaatagttgtaagggttccatcatgcacttaatatccctgtgggcctattgatatttccataattcattctcgccttcctatatccctttcttgttataatgatttatataagcgctcttcaaggaggaacatccctaacatgaggattacccatgtaactaactaagtttaaacactaaccaaaagctgggcgagtcagatgaggttcataatgttacacgacagagaaagctcagtgcctgagtgagatacttttgtagattaagtgcatgatggaacccttacaactattgtacatctaagataggaaggcgagaatgaattatggaaatatcaataggcccacgttcctccttgaagagcgcttatataaatcattataacaagaattaattaataacattatgaacctcatctgactcgcccaggccggcatggtcccagcctcctcgctggcgccggctgggcaacattcgtggcgaatgggaccatagcggccgc

SEQ ID NO: 6 - DNA, 662 bp, Artificial Sequence
  DNA sequence designed for transcription into an RNA.
  misc_feature (1)..(8): AsiSI restriction site.
  misc_feature (9)..(62): Hammerhead ribozyme cuts between residues 62-63.
  misc_feature (63)..(75): antisense to sequence between residues 324-336.
  misc_feature (82)..(126): antisense to sequence between residues 369-413.
  misc_feature (133)..(177): antisense to sequence between residues 414-458.
  misc_feature (184)..(228): antisense to sequence between residues 459-503.
  misc_feature (235)..(279): antisense to sequence between residues 504-548.
  misc_feature (287)..(305): bacteriophage MS2 PAC site.
  misc_feature (316)..(323): PmeI restriction site.
  misc_feature (316)..(323): PmeI restriction site.
  misc_feature (324)..(425): filler sequence.
  misc_feature (426)..(446): sequence no. 21.3 of Bolognesi, et al., (2012) PLoS One 7e47534.
  misc_feature (447)..(548): filler sequence.
  misc_feature (549)..(556): PacI restriction site.
  misc_feature (557)..(588): antisense to sequence between residues 337-368.
  misc_feature (589)..(654): HDV ribozyme, cuts between residues 568 and 569.
  misc_feature (655)..(662): NotI restriction site.
gcgatcgcataggatactaaccactgatgaggccgaaaggccgaaacccgtgaaaacgggtctggttagtatcctatcccttacaaaagtatctcactcaggcactgagctttctctgtcgtgtaaatcccttcttagatgtacaatagttgtaagggttccatcatgcacttaatcatccctaacgtgggcctattgatatttccataattcattctcgccttcctaatcccttgtccttcttgttataatgatttatataagcgctcttcaaggaggatccctaacatgaggattacccatgtaactaactaagtttaaacaggatactaaccaaaagctgggcgagtcagatgaggttcataatgttacacgacagagaaagctcagtgcctgagtgagatacttttgtagattaagtgcatgatggaacccttacaactattgtacatctaagataggaaggcgagaatgaattatggaaatatcaataggcccacgttcctccttgaagagcgcttatataaatcattataacaagaaggacattaattaacattatgaacctcatctgactcgcccagctttggccggcatggtcccagcctcctcgctggcgccggctgggcaacattcgtggcgaatgggaccatagcggccgc

SEQ ID NO: 7 - DNA, 682 bp, Artificial Sequence
  DNA sequence designed for transcription into an RNA.
  misc_feature (1)..(8): AsiSI restriction site.
  misc_feature (9)..(62): Hammerhead ribozyme cuts between residues 62-63.
  misc_feature (63)..(75): antisense to sequence between residues 332-344.
  misc_feature (82)..(128): antisense to sequence between residues 379-425.
  misc_feature (135)..(181): antisense to sequence between residues 426-472.
  misc_feature (188)..(234): antisense to sequence between residues 473-519.
  misc_feature (241)..(287): antisense to sequence between residues 520-560.
  misc_feature (295)..(313): bacteriophage MS2 PAC site.
  misc_feature (324)..(331): PmeI restriction site.
  misc_feature (332)..(438): filler sequence.
  misc_feature (439)..(459): sequence no. 21.3 of Bolognesi, et al., (2012) PLoS One 7e47534.
  misc_feature (460)..(566): filler sequence.
  misc_feature (567)..(574): PacI restriction site
  misc_feature (575)..(608): antisense to sequence between residues 345-378.
  misc_feature (609)..(674): HDV ribozyme, cuts between residues 608 and 609.
  misc_feature (675)..(682): NotI restriction site.
gcgatcgcatcaaagaggatactctgatgaggccgaaaggccgaaacccgtgaaaacgggtcagtatcctctttgatccctacaaaagtatctcactcaggcactgagctttctctgtcgtgtaacatatccctatcttagatgtacaatagttgtaagggttccatcatgcacttaatctatccctaggaacgtgggcctattgatatttccataattcattctcgccttcctatccctcgccttgtccttcttgttataatgatttatataagcgctcttcaaggatccctaacatgaggattacccatgtaactaactaagtttaaaccaaagaggatactaaccaaaagctgggcgagtcagatgaggttcataatgttacacgacagagaaagctcagtgcctgagtgagatacttttgtagattaagtgcatgatggaacccttacaactattgtacatctaagataggaaggcgagaatgaattatggaaatatcaataggcccacgttcctccttgaagagcgcttatataaatcattataacaagaaggacaaggcgttaattaatatgaacctcatctgactcgcccagcttttggttggccggcatggtcccagcctcctcgctggcgccggctgggcaacattcgtggcgaatgggaccatagcggccgc

SEQ ID NO: 8 - DNA, 702 bp, Artificial Sequence
  DNA sequence designed for transcription into an RNA
  misc_feature (1)..(8): AsiSI restriction iste.
  misc_feature (9)..(62): Hammerhead ribozyme cuts between residues 62-63.
  misc_feature (63)..(75): antisense to sequence between residues 340-352.
  misc_feature (82)..(130): antisense to sequence between residues 389-437.
  misc_feature (137)..(185): antisense to sequence between residues 438-486.
  misc_feature (192)..(240): antisense to sequence between residues 487-535.
  misc_feature (247)..(295): antisense to sequence between residues 536-584.
  misc_feature (303)..(321): bacteriophage MS2 PAC site.
  misc_feature (332)..(339): PmeI restriction site.
  misc_feature (340)..(438): filler sequence.
  misc_feature (452)..(472): sequence no. 21.3 of Bolognesi, et al., (2012) PLoS One 7e47534.
  misc_feature (473)..(584): filler sequence.
  misc_feature (585)..(592): PacI restriction site.
  misc_feature (593)..(628): antisense to sequence between residues 353-388.
  misc_feature (629)..(694): HDV ribozyme, cuts between residues 628 and 629.
  misc_feature (695)..(702): NotI restriction site.
gcgatcgcataatgccaaagaggctgatgaggccgaaaggccgaaacccgtgaaaacgggtccctctttggcattatccctcaaaagtatctcactcaggcactgagctttctctgtcgtgtaacattatatcccttatcttagatgtacaatagttgtaagggttccatcatgcacttaatctaatccctaggaggaacgtgggcctattgatatttccataattcattctcgccttccatccctgcattcgccttgtccttcttgttataatgatttatataagcgctcttcaatccctaacatgaggattacccatgtaactaactaagtttaaacaatgccaaagaggatactaaccaaaagctgggcgagtcagatgaggttcataatgttacacgacagagaaagctcagtgcctgagtgagatacttttgtagattaagtgcatgatggaacccttacaactattgtacatctaagataggaaggcgagaatgaattatggaaatatcaataggcccacgttcctccttgaagagcgcttatataaatcattataacaagaaggacaaggcgaatgcttaattaagaacctcatctgactcgcccagcttttggttagtatggccggcatggtcccagcctcctcgctggcgccggctgggcaacattcgtggcgaatgggaccatagcggccgc

SEQ ID NO: 9 - DNA, 793 bp, Artificial Sequence
  DNA sequence designed for transcription into RNA.
  misc_feature (1)..(8): AsiSI rest5iction site.
  misc_feature (9)..(61): Hammerhead ribozyme cuts between residues 61-62.
  misc_feature (62)..(296): antisense to sequence between residues 455-689.
  misc_feature (297)..(304): PmeI restriction site.
  misc_feature (305)..(454): loop sequence.
  misc_feature (455)..(561): filler sequence.
  misc_feature (562)..(582): sequence no. 21.3 of Bolognesi, et al., (2012) PLoS One 7e47534.
  misc_feature (583)..(689): filler sequence.
  misc_feature (691)..(709): bacteriophage MS2 PAC site.
  misc_feature (720)..(785): HDV ribozyme, cuts between residues 719 and 720.
  misc_feature (786)..(793): NotI restriction site.
gcgatcgcggcgaccctgatgaggccgaaaggccgaaacccgtgaaaacgggtcggtcgcccgccttgtccttcttgttataatgatttatataagcgctcttcaaggaggaacgtgggcctattgatatttccataattcattctcgccttcctatcttagatgtacaatagttgtaagggttccatcatgcacttaatctacaaaagtatctcactcaggcactgagctttctctgtcgtgtaacattatgaacctcatctgactcgcccagcttttggttagtatcctctttggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctcaaagaggatactaaccaaaagctgggcgagtcagatgaggttcataatgttacacgacagagaaagctcagtgcctgagtgagatacttttgtagattaagtgcatgatggaacccttacaactattgtacatctaagataggaaggcgagaatgaattatggaaatatcaataggcccacgttcctccttgaagagcgcttatataaatcattataacaagaaggacaaggcgaacatgaggattacccatgtaactaactaaggccggcatggtcccagcctcctcgctggcgccggctgggcaacattcgtggcgaatgggaccatagcggccgc